(19) United States
(12) Patent Application Publication
(10) Pub. No.: US 2003/0086873 A1
(43) Pub. Date: May 8, 2003

(54) Myocardial imaging agent and preparation method thereof

(76) Inventors: Bo Li Liu, Beijing (CN); Yan Jiang, Beijing (CN)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/133,245

(22) Filed: Apr. 26, 2002

(30) Foreign Application Priority Data
Apr. 27, 2001 (CN) 01115464.0

(51) Int. Cl. A61B 5/055

(52) U.S. Cl. 424/9.3

(57) ABSTRACT

The invention provides tricarbonyl coordination complexes of having the formula:

[M(CO)₃(MIBI)ₓ(OH₂)₃₋ₓ]⁺,

wherein M is a radioactive isotope selected from the group consisting of Mn, ^{99m}Tc, ¹⁸⁶Re, and ¹⁸⁸Re, MIBI is the ether isonitrile 2-methoxy isobutylisonitrile (CN—CH₂—C(CH₃)₂—OCH₃), and wherein x=1 to 3. The compositions are useful as myocardial imaging agents. The compositions of the invention exhibit fast cardiac uptake, fast lung clearance, and low liver background. The invention also provides methods for preparing the tricarbonyl coordination complexes, method for preparing the coordination complex [M(CO)₃(OH₂)₃]⁺, radiopharmaceutical compositions comprising the tricarbonyl coordination complexes, and methods for imaging body tissues using the tricarbonyl coordination complexes.

### RELATED APPLICATIONS

This application claims priority under 35 U.S.C. §119(a) to Chinese Patent Application No. 01115464.0, filed Apr. 27, 2001, entitled “Myocardial Imaging Agent and Preparation Method Thereof.”

### FIELD OF THE INVENTION

This invention is directed to organometallic coordination complexes that are useful as agents for imaging body tissues, particular for imaging myocardial tissue. The invention is also directed to methods for preparing said organometallic complexes, methods for preparing intermediates used in the preparation of organometallic complexes, radiopharmaceutical compositions comprising organometallic complexes, and methods for imaging body tissues using organometallic complexes.

### BACKGROUND OF THE INVENTION

Coordination complexes of isonitrile ligands (CN-R, wherein R is an organic radical) and radionuclides have proven to be useful myocardial perfusion imaging agents. See U.S. Pat. No. 4,452,774, incorporated herein by reference in its entirety. One complex that is particularly useful for myocardial imaging is the unipositively charged hexakis ^{99m}Tc complex [^{99m}Tc(MIBI)₆]⁺, wherein MIBI is the ether isonitrile 2-methoxy isobutyl isonitrile (CN—CH₂—C(CH₃)₂—OCH₃). See U.S. Pat. No. 4,988,827, incorporated herein by reference in its entirety. This complex is also known as ^{99m}Tc-sestamibi; a kit for its production is marketed by Du Pont under the name CARDIOLITE®. Despite its widespread use, [^{99m}Tc(MIBI)₆]⁺ is not an ideal cardiac imaging agent due to the presence of a high liver background. The resulting liver shadow affects the image of part of the cardiac apex (Journal of Nuclear Medicine, 1989, 30:801-811; International Journal of Nuclear Medicine and Biology, 1984, 11:225-234, both incorporated herein by reference in their entirety). Given this problem, it would be desirable to have radionuclide coordination complexes that exhibit higher myocardial concentrations and higher heart:liver uptake ratios than [^{99m}Tc(MIBI)₆]⁺.

### SUMMARY OF THE INVENTION

The invention provides tricarbonyl coordination complexes having the formula:

[M(CO)₃(MIBI)ₓ(OH₂)₃₋ₓ]⁺

wherein:

M is a radioactive isotope selected from the group consisting of Mn, ^{99m}Tc, ¹⁸⁶Re, and ¹⁸⁸Re;

MIBI is 2-methoxy isobutylisonitrile; and

x=1 to 3.

A particularly preferred complex is [^{99m}Tc(CO)₃(MIBI)₃]⁺. The compositions are useful as myocardial imaging agents. In comparison to [^{99m}Tc(MIBI)₆]⁺, the compositions of the invention exhibit unexpectedly superior properties, namely: faster cardiac uptake, faster lung clearance, and lower liver background.

The invention also provides a method for preparing a tricarbonyl coordination complex having the formula:

[M(CO)₃(MIBI)ₓ(OH₂)₃₋ₓ]⁺

wherein:

M is a radioactive isotope selected from the group consisting of Mn, ^{99m}Tc, ¹⁸⁶Re, and ¹⁸⁸Re;

MIBI is 2-methoxy isobutylisonitrile;

x=1 to 3;

the method comprising reacting a predetermined quantity of [M(CO)₃(OH₂)₃]⁺ in a mixture with a predetermined quantity of MIBI.

The invention also includes a method for preparing a tricarbonyl coordination complex having the formula:

[M(CO)₃(OH₂)₃]⁺

wherein:

M is a radioactive isotope selected from the group consisting of Mn, ^{99m}Tc, ¹⁸⁶Re, and ¹⁸⁸Re;

comprising reacting M in the permetallate form in a mixture with carbon monoxide, borohydride ion, carbonate ion, and tartrate ion.

The invention further includes a method for imaging body tissues comprising: administering to an animal a radiopharmaceutical composition comprising a tricarbonyl coordination complex having the formula:

[M(CO)₃(MIBI)ₓ(OH₂)₃₋ₓ]⁺,

wherein:

M is a radioactive isotope selected from the group consisting of Mn, ^{99m}Tc, ¹⁸⁶Re, and ¹⁸⁸Re;

MIBI is 2-methoxy isobutylisonitrile;

x=1 to 3;

and detecting the localization of such complex in the body tissues by a gamma camera.

The invention also includes radiopharmaceutical compositions comprising:

a tricarbonyl coordination complex having the formula:

[M(CO)₃(MIBI)ₓ(OH₂)₃₋ₓ]⁺

wherein:

M is a radioactive isotope selected from the group consisting of Mn, ^{99m}Tc, ¹⁸⁶Re, and ¹⁸⁸Re;

MIBI is 2-methoxy isobutylisonitrile;

x=1 to 3;

and one or more pharmaceutically accepted excipients or diluents.

The compositions and methods of the instant invention enable one to obtain clear images of the myocardium without the impact of liver shadow.

### DETAILED DESCRIPTION OF THE INVENTION

The tricarbonyl coordination complexes of the invention have the formula [M(CO)₃(MIBI)ₓ(OH₂)₃₋ₓ]⁺, wherein M is a radioactive isotope selected from the group consisting of Mn, ^{99m}Tc, ¹⁸⁶Re, and ¹⁸⁸Re, MIBI is the ether isonitrile 2-methoxy isobutylisonitrile (CN—CH₂—C(CH₃)₂—OCH₃), and wherein x=1 to 3. The complexes are preferably made by the addition of MIBI to [M(CO)₃(OH₂)₃]⁺, most preferably at elevated temperature and under reducing conditions.

[M(CO)₃(OH₂)₃]⁺ is preferably prepared by introducing carbon monoxide (CO) gas into a permanganate (when M=Mn), pertechnetate (when M=^{99m}Tc), or perrhenate (when M=¹⁸⁶Re or ¹⁸⁸Re) solution under reducing conditions, preferably at elevated temperature. The preferred reducing agent is the borohydride anion. Other methods for the preparation of [M(CO)₃(OH₂)₃]⁺ are provided in WO 98/48848, EP 0 879 606, and Coordination Chemistry Reviews, 1999, 190-192: 901-919, each of which is incorporated herein by reference in its entirety.

In one embodiment, [^{99m}Tc(CO)₃(OH₂)₃]⁺ is prepared in the following manner. First, sodium borohydride (preferably about 10 mg to about 30 mg), sodium carbonate (preferably about 10 mg to about 20 mg), and potassium-sodium tartrate (about 10 mg to about 20 mg) are placed into suitable container, such as a flask. The container is sealed, and carbon monoxide gas is passed through for period of time sufficient to purge air (preferably from about 10 minutes to about 20 minutes). Then sodium pertechnetate solution (preferably about 1 mL, and preferably its highest activity being 3×10¹⁰Bq) and buffer solution (preferably about 1 mL, and preferably with pH of about 10 to about 12) is added thereto. The reaction is allowed to proceed and carbon monoxide gas is continuously passed through the solution throughout the entire reaction method. Preferably the reaction is allowed to proceed for about 10 minutes to about 40 minutes at about 60° C. to about 80° C. When the reaction is complete, the solution is cooled down to room temperature and a buffer solution (preferably phosphate buffered saline, most preferably comprising a mixture of about 0.05 to about 0.3 mol/L of sodium chloride and about 0.01 to about 0.2 mol/L of phosphate) is added thereto to a adjust the pH value to between about pH 6 and about pH 8. It will be appreciated that the foregoing method can be scaled up or down in size (while retaining approximately the same molar ratios of the reagents). In addition, it will be appreciated that the method can also be carried out using permanganate or perrhenate instead of pertechnetate.

The resulting water-soluble organotechnetium cation [M(CO)₃(OH₂)₃]⁺ is quite stable in water and air, and the water (OH₂) ligand can be easily replaced with other ligands with a higher coordination capacity, which makes it possible for the carbonyl complex to be used in nuclear medicine as a radioactive drug. The complex compound [^{99m}Tc(CO)₃(OH₂)₃]⁺ is described in Coordination Chemistry Reviews, 1999, 190-192: 901-919, and in Nature Biotechnology 1999, 17: 897-901.

The unipositively charged tricarbonyl complexes according to the formula [M(CO)₃(MIBI)ₓ(OH₂)₃₋ₓ]⁺ may be prepared by adding MIBI (2-methoxy isobutyl isonitrile) to [M(CO)₃(OH₂)₃]⁺ (preferably prepared according to the preceeding embodiment). The MIBI is preferably added in freeze-dried form. Preferably, one or more reducing agents are also added to the reaction mixture. A preferred reducing agent is L-cysteine. Mannitol and glucose are also preferably added to the reaction mixture. When MIBI is added to the [M(CO)₃(OH₂)₃]⁺ prepared in the preferred volume as described in the foregoing embodiment, the preferred quantity of L-cysteine is about 50 μg to about 100 μg; the preferred quantity of mannitol is about 10 mg to about 20 mg; and the preferred quantity of glucose is about 10 mg to about 20 mg. The reaction mixture is preferably heated to about 80° C. up to about 100° C. for a period of time between about 20 minutes and about 45 minutes.

The tricarbonyl complexes of the instant invention are useful as myocardial imaging agents. The complex [^{99m}Tc(CO)₃(MIBI)₃]⁺ is particularly preferred as a myocardial imaging agent due to its rapid lung clearance and high heart:liver uptake ratio. For myocardial imaging, the tricarbonyl complexes are preferably injected intravenously. The radiopharmaceutical composition that is injected comprises the tricarbonyl complex [M(CO)₃(MIBI)ₓ(OH₂)₃₋]⁺ in a pharmaceutically acceptable excipient or diluent. Preferably, imaging is performed within about 10 to about 120 minutes from the time of injection; however, imaging is possible both before and after this preferred time window. The tricarbonyl complexes are useful for a variety of imaging techniques, including, but not limited to, flat and tomographic myocardial imaging with gamma cameras.

The following examples are for provided for illustrative purposes only. They are not to be interpreted as limiting the scope of the invention in any way.

### EXAMPLES

### Example 1

Preparation of the Tricarbonyl Complex [^{99m}Tc(CO)₃(MIBI)ₓ(OH₂)₃₋ₓ]⁺

First, 10 mg of sodium borohydride, 10 mg of sodium carbonate, and 16 mg of potassium-sodium tartrate were placed into a small 25 ml flask with a circular bottom. The flask was sealed and carbon monoxide gas was passed through for 10 minutes to purge the air from the flask. Then 1 ml of sodium pertechnetate (Na^{+99m}TcO₄⁻) rinsing solution (its highest activity is 30 GBq) and 1 ml of buffer solution with pH of 10.8 were added thereto using an injector. The reaction was allowed to proceed for 25 minutes at 75° C., and throughout the entire reaction method carbon monoxide gas was continuously passed through the solution. Once the reaction had concluded, the solution was cooled down to room temperature and a PBS buffer solution (phosphate buffered saline comprising a mixture of 0.1 mol/L of sodium chloride and 0.05 mol/L of phosphate) was added thereto to adjust the pH value to 7, thus obtaining [^{99m}Tc(CO)₃(OH₂)₃]⁻. Then, 1 mg of freeze-dried MIBI (2-methoxy isobutyl isonitrile with additives (75 μg of L-cysteine, 10 mg of mannitol, and 10 mg of glucose) was added directly into the [^{99m}Tc(CO)₃(OH₂)₃]⁺. The reaction was allowed to proceed for 35 min at 90° C., at which time the desired product, comprising mainly [^{99m}Tc(CO)₃(MIBI)₃]⁺ was obtained. The reaction product was analyzed using thin layer chromatography, with a polyamide thin plate as the stationary phase. The developing solvents are included in Table 1, and the radiochemical yield of both products was as high as 85%.

TABLE 1
Developing Solvents used in Thin-Layer Chromatography and
R₆ Values of the Products

| | R₆ Value of | R₆ Value of |
|---|---|---|
| Developing Solvent | [^{99m}Tc(CO)₃(OH₂)₃]⁺ | [^{99m}Tc(CO)₃(MIBI)₃]⁺ |
| Acetonitrile | 0.1-0.2 | 0.9-1.0 |
| 0.9% normal saline: | 0.7-1.0 | 0.3-0.5 |
| acetone: concentrated | | |
| ammonia water (9:2:0.1) | | |

### Example 2

Comparison of the Results of Biological Distribution of [^{99m}Tc(CO)₃(OH₂)₃]⁺, [^{99m}Tc(CO)₃(MIBI)₃]⁺, [^{99m}Tc(MIBI)₆]+ in the Bodies of Kunming Mice

The complexes [^{99m}Tc(CO)₃(OH₂)₃]⁺ and [^{99m}Tc(CO)₃(MIBI)₃]⁺ prepared in Example 1 were separately injected in the caudal vein of Kunming mice. The experiment was conducted on three mice within each time period, and the injection's activity was 925 KBq. The radioactivity of individual organs's tissue was measured at 5, 15, 30, and 60 minutes after the injection, and the results are represented by radioactive uptake rate per gram of tissue.

There was no specific concentration of [^{99m}Tc(CO)₃(OH₂)₃]⁺ (radiochemical yield of 85%) in any of the tissues, but they displayed good radioactive retention within 60 minutes. There was an apparent concentration of [^{99m}Tc(CO)₃(MIBI)₃]⁺ (radiochemical yield of 85%) in the mice's hearts with a good radioactive retention, while its concentration in their livers was comparatively low. The results of the distribution of [^{99m}Tc(CO)₃(OH₂)₃]⁺ in the mice's bodies are included in Tables 2; the results for [^{99m}Tc(CO)₃(MIBI)₃]⁺ are presented in Table 3. The value “% ID/g” is the percentage of the injection's total radioactivity that each gram of tissue accounts for i.e., the radioactive uptake rate per gram of tissue, with ID standing for “injected dose.”

TABLE 2
Biological Distribution of [^{99m}Tc(CO)₃(OH₂)₃]⁺ in the Bodies of
Mice (% ID/g, average value ± standard deviation, n = 3)

| Tissue | 5 Minutes | 15 Minutes | 30 Minutes | 60 Minutes |
|---|---|---|---|---|
| Heart | 2.68 ± 0.88 | 2.39 ± 0.66 | 2.39 ± 0.56 | 2.08 ± 0.31 |
| Liver | 10.33 ± 0.82 | 11.95 ± 0.66 | 10.83 ± 1.68 | 10.73 ± 0.90 |
| Lungs | 6.53 ± 1.41 | 5.38 ± 0.76 | 5.37 ± 0.78 | 5.19 ± 1.18 |
| Blood | 9.27 ± 1.13 | 6.04 ± 0.73 | 5.79 ± 0.66 | 4.50 ± 0.72 |
| Kidneys | 18.29 ± 4.22 | 14.25 ± 1.78 | 13.88 ± 1.02 | 12.28 ± 2.32 |
| Brain | 0.31 ± 0.09 | 0.27 ± 0.03 | 0.29 ± 0.06 | 0.22 ± 0.03 |
| Muscles | 1.47 ± 0.37 | 1.46 ± 0.48 | 1.66 ± 0.61 | 1.37 ± 0.80 |
| Bones | 2.82 ± 0.95 | 1.68 ± 0.22 | 2.38 ± 0.54 | 1.65 ± 0.12 |
| Spleen | 2.02 ± 1.02 | 2.38 ± 0.37 | 2.94 ± 0.69 | 3.20 ± 0.80 |

TABLE 3
Biological Distribution of the [^{99m}Tc(CO)₃(MIBI)₃]⁺ Complex with
Positive Valence 1 in the Bodies of Mice
(% ID/g, average value ± standard deviation, n = 3)

| Tissue | 5 Minutes | 15 Minutes | 30 Minutes | 60 Minutes |
|---|---|---|---|---|
| Heart | 21.62 ± 2.84 | 20.63 ± 3.89 | 20.77 ± 1.60 | 19.39 ± 0.91 |
| Liver | 11.48 ± 0.88 | 8.28 ± 0.90 | 6.44 ± 1.03 | 4.69 ± 0.34 |
| Lungs | 6.16 ± 0.48 | 4.14 ± 0.90 | 3.89 ± 0.54 | 3.20 ± 0.85 |
| Blood | 1.03 ± 0.21 | 0.32 ± 0.05 | 0.24 ± 0.07 | 0.13 ± 0.04 |
| Kidneys | 57.90 ± 1.80 | 33.66 ± 0.42 | 23.94 ± 2.11 | 11.30 ± 0.73 |
| Brain | 0.18 ± 0.02 | 0.18 ± 0.02 | 0.12 ± 0.01 | 0.08 ± 0.01 |
| Muscles | 7.08 ± 0.52 | 3.78 ± 0.88 | 6.14 ± 0.78 | 4.42 ± 0.36 |
| Bones | 3.72 ± 0.84 | 2.10 ± 0.10 | 1.94 ± 0.29 | 1.42 ± 0.31 |
| Spleen | 6.26 ± 1.29 | 3.84 ± 0.90 | 2.84 ± 0.41 | 1.62 ± 0.32 |

[^{99m}Tc(CO)₃(MIBI)₃]⁺ has a higher heart:liver ratio in the mice's bodies than [^{99m}Tc(MIBI)₆]⁺ and a comparable heart:blood ratio. The ratios of the racioactive uptake of the complexes in the heart and in the other contiguous tissues are presented in Tables 4 and 5.

TABLE 4
Target/Non-Target Uptake Ratio Values of [^{99m}Tc(CO)₃(MIBI)₃]⁺

| Target/ | | | | |
|---|---|---|---|---|
| Non-Target | 5 Minutes | 15 Minutes | 30 Minutes | 60 Minutes |
| Heart/Liver | 1.88 | 2.49 | 3.23 | 4.13 |
| Heart/Lungs | 3.51 | 4.98 | 5.34 | 6.06 |
| Heart/Blood | 20.99 | 64.47 | 86.54 | 149.15 |

TABLE 5
Target/Non-Target Uptake Ratio Values of [^{99m}Tc(MIBI)₆]⁺

| Target/ | | | | |
|---|---|---|---|---|
| Non-Target | 5 Minutes | 15 Minutes | 30 Minutes | 60 Minutes |
| Heart/Liver | 0.94 | 0.76 | 0.90 | 1.04 |
| Heart/Lungs | 2.87 | 6.08 | 9.07 | 12.25 |
| Heart/Blood | 12.80 | 35.09 | 89.04 | 134.15 |

### Example 3

Pharmacological Experimental Study of [^{99m}Tc(CO)₃(MIBI)₃]³⁰ in Dogs, and Comparison with [^{99m}Tc(MIBI)₆]⁺

Dogs were intravenously injected with [^{99m}Tc(CO)₃(MIBI)₃]⁺ or [^{99m}Tc(MIBI)₆]⁻at dose of 5.55×10⁵ Bq. Immediately following injection, dynamic photography for up to 120 min was performed using a Toshiba GCA 7200A two-probe SPECT and a low energy parallel aperture regular collimator. Time—radioactivity curves were also collected for the heart, liver, lungs, and kidneys. Target/non-target radioactivity ratios were calculated, and fall body imaging was performed separately at different times. The biological distributions of the imaging agents in the body were analyzed. Vein blood samples were collected separately at different times, and the dynamics of the blood clearance was analyzed. Flat myocardial imaging was conducted separately at different times, with myocardial tomographic imaging at 120 minutes post injection. The results demonstrated that the dogs' blood clearance curve matched the secondary model of drug metabolic kinetics for primary intravenous administration (T(α)_{⅓}=1.33±0.12minutes, T(β)_{½}=102.33±25.58 minutes, CL=401.33±73.51 mL/hour)

The time—radioactivity curves for heart, liver, lungs, and kidneys demonstrated that for [^{99m}Tc(CO)₃(MIBI)₃]⁺ the liver curve is lower than the myocardial curve, while for [^{99m}Tc(MIBI)₆]⁺ the liver curve is higher than the myocardial curve. The biological distribution of [^{99m}Tc(CO)₃(MIBI)₃]⁺ in the body, the ratio of target/non-target uptake, and comparisons with the same measurements for [^{99m}Tc(MIBI)₆]⁺ are included in Tables 6-9.

The flat and tomagraphic images of the myocardium demonstrated that a distinct image of the myocardium can be obtained at any moment within the period between 10 and 120 minutes after the intravenous injection of [^{99m}Tc(CO)₃(MIBI)₃]⁺. The radioactivity in the liver was clearly lower than with [^{99m}Tc(MIBI)₆]⁺. [^{99m}Tc(CO)₃(MIBI)₃]⁺ has the advantages of fast myocardial uptake, fast lung clearance, and low liver background. [^{99m}Tc(CO)₃(MIBI)₃]⁺ is an unexpectedly superior myocardial perfusion imaging agent in comparison with [^{99m}Tc(MIBI)₆]⁻.

TABLE 6
Biological Distribution of [^{99m}Tc(CO)₃(MIBI)₃]⁺ in the Bodies of Dogs (% ID/g, average value ± standard deviation, n = 3)

| Tissue | 10 Minutes | 20 Minutes | 30 Minutes | 40 Minutes | 60 Minutes | 90 Minutes | 120 Minutes |
|---|---|---|---|---|---|---|---|
| Heart | 3.80 ± 0.44 | 3.97 ± 0.50 | 4.00 ± 0.30 | 3.73 ± 0.51 | 3.63 ± 0.49 | 3.77 ± 0.64 | 3.73 ± 0.58 |
| Lungs | 5.13 ± 0.06 | 4.70 ± 0.20 | 4.70 ± 0.26 | 4.80 ± 0.72 | 4.40 ± 0.44 | 4.20 ± 0.26 | 4.07 ± 0.15 |
| Liver | 12.93 ± 0.70 | 14.00 ± 1.78 | 14.27 ± 1.97 | 16.37 ± 2.47 | 17.53 ± 2.85 | 19.67 ± 3.72 | 21.03 ± 2.78 |
| Kidneys | 15.33 ± 0.75 | 14.70 ± 1.14 | 14.53 ± 1.86 | 13.27 ± 1.65 | 12.23 ± 1.00 | 12.37 ± 1.75 | 12.40 ± 3.48 |

TABLE 7
Biological Distribution of
[^{99m}Tc(MIBI)₆]⁺ in the Bodies of Dogs (% ID/g), n = 1

| | 20 | 30 | 40 | 60 | 90 | 120 |
|---|---|---|---|---|---|---|
| Tissue | Minutes | Minutes | Minutes | Minutes | Minutes | Minutes |
| Heart | 3.2 | 3.5 | 2.9 | 3.3 | 3.2 | 3.5 |
| Lungs | 6.3 | 6.5 | 6.3 | 5.5 | 6.2 | 5.2 |
| Liver | 21.0 | 10.3 | 24.4 | 25.1 | 22.8 | 25.2 |
| Kidneys | 14.3 | 3.2 | 12.3 | 12.7 | 13.2 | 12.1 |

TABLE 8
Target/Non-Target Uptake Ratio Values of [^{99m}Tc(CO)₃(MIBI)₃]⁺ (% ID/g, average value ± standard deviation, n = 3)

| Tissue | 2 Minutes | 5 Minutes | 10 Minutes | 20 Minutes | 30 Minutes | 40 Minutes | 60 Minutes | 90 Minutes | 120 Minutes |
|---|---|---|---|---|---|---|---|---|---|
| Heart/ | 2.51 ± 0.25 | 2.67 ± 0.27 | 2.60 ± 0.26 | 2.80 ± 0.44 | 2.96 ± 0.43 | 3.00 ± 0.51 | 3.05 ± 0.52 | 3.10 ± 0.49 | 3.09 ± 0.56 |
| Lungs | | | | | | | | | |
| Heart/ | 1.00 ± 0.18 | 0.98 ± 0.11 | 0.92 ± 0.13 | 0.97 ± 0.10 | 1.02 ± 0.11 | 1.06 ± 0.17 | 1.22 ± 0.20 | 1.25 ± 0.33 | 1.37 ± 0.26 |
| Liver | | | | | | | | | |
| Heart/ | 0.56 ± 0.13 | 0.53 ± 0.12 | 0.53 ± 0.10 | 0.59 ± 0.10 | 0.62 ± 0.15 | 0.58 ± 0.08 | 0.60 ± 0.06 | 0.60 ± 0.06 | 0.63 ± 0.04 |
| Kidneys | | | | | | | | | |

TABLE 9
Target/Non-Target Uptake Ratio Values of [^{99m}Tc(MIBI)₆]⁺ (% ID/g), n = 1

| Tissue | 2 Minutes | 5 Minutes | 10 Minutes | 20 Minutes | 30 Minutes | 40 Minutes | 60 Minutes | 90 Minutes | 120 Minutes |
|---|---|---|---|---|---|---|---|---|---|
| Heart/ | 2.35 | 2.48 | 2.66 | 2.80 | 2.68 | 2.70 | 2.84 | 2.84 | 2.73 |
| Lungs | | | | | | | | | |
| Heart/ | 0.83 | 0.83 | 0.77 | 0.85 | 0.86 | 0.87 | 0.84 | 0.80 | 0.80 |
| Liver | | | | | | | | | |
| Heart/ | 0.39 | 0.48 | 0.59 | 0.45 | 0.43 | 0.43 | 0.43 | 0.46 | 0.45 |
| Kidneys | | | | | | | | | |

What is claimed is:

1. A tricarbonyl coordination complex having the formula:
[M(CO)₃(MIBI)ₓ(OH)₂)_{3-x]}⁺
wherein:
M is a radioactive isotope selected from the group consisting of Mn, ^{99m}Tc, ¹⁸⁶Re, and ¹⁸⁸Re;
MIBI is 2-methoxy isobutylisonitrile; and
x=1 to 3.

2. The complex of claim 1 which is [^{99m}Tc(CO)₃(MIBI)₃]⁺.

3. The complex of claim 1 which is [^{99m}Tc(CO)₃(MIBI)₂(OH₂)]⁺.

4. The complex of claim 1 which is [^{99m}Tc(CO)₃(MIBI)(OH₂)₂]⁺.

5. A method for preparing a tricarbonyl coordination complex having the formula:
[M(CO)₃(MIBI)ₓ(OH₂)₃₋ₓ]⁺
wherein:
M is a radioactive isotope selected from the group consisting of Mn, ^{99m}Tc, ¹⁸⁶Re, and ¹⁸⁸Re;
MIBI is 2-methoxy isobutyl isonitrile; and
x=1 to 3;
the method comprising reacting a predetermined quantity of [M(CO)₃(OH₂)₃]⁺ in a mixture with a predetermined quantity of MIBI.

6. The method of claim 5 wherein said tricarbonyl coordination complex is [^{99m}Tc(CO)₃(MIBI)₃]⁺.

7. The method of claim 5 wherein said predetermined quantity of MIBI is freeze-dried.

8. The method of claim 5 wherein the reaction takes place under reducing conditions.

9. The method of claim 8 wherein said reducing conditions are achieved by adding L-cysteine to the reaction.

10. The method of claim 5 wherein the reaction occurs at a temperature between about 80° C. and about 100° C. for a period of time between about 20 minutes and about 45 minutes.

11. The method of claim 5 wherein a predetermined quantity of mannitol is added to said mixture.

12. The method of claim 5 wherein a predetermined quantity of glucose is added to said mixture.

13. A method for preparing a tricarbonyl coordination complex having the formula:
[M(CO)₃(OH₂)₃]⁺
wherein M is a radioactive isotope selected from the group consisting of Mn, ^{99m}Tc, ¹⁸⁶Re, and ¹⁸⁸Re;
comprising reacting M in the permetallate form in a mixture with carbon monoxide, borohydride ion, carbonate ion, and tartrate ion.

14. A method for imaging body tissues comprising:
administering to an animal a radiopharmaceutical composition comprising a tricarbonyl coordination complex having the formula:
[M(CO)₃(MIBI)ₓ(OH₂)₃₋ₓ]⁺,
wherein:
M is a radioactive isotope selected from the group consisting of Mn, ^{99m}Tc, ¹⁸⁶Re, and ¹⁸⁸Re;
MIBI is 2-methoxy isobutylisonitrile;
x=1 to 3;
and detecting the localization of such complex in the body tissues by a gamma camera.

15. The method of claim 14 wherein said tricarbonyl complex is [^{99m}Tc(CO)₃(MIBI)₃]⁺.

16. The method of claim 14 wherein said tricarbonyl complex is administered intravenously.

17. The method of claim 14 wherein said body tissue imaged is myocardial tissue.

18. The method of claim 14 wherein said imaging is tomographic.

19. The method of claim 14 wherein said imaging is flat.

20. A radiopharmaceutical composition comprising:
a tricarbonyl coordination complex having the formula:
[M(CO)₃(MIBI)ₓ(OH₂)₃₋ₓ]+
wherein:
M is a radioactive isotope selected from the group consisting of Mn, ^{99m}Tc, ¹⁸⁶Re, and ¹⁸⁸Re;
MIBI is 2-methoxy isobutylisonitrile;
x=1 to 3;
and one or more pharmaceutically accepted excipients or diluents.

21. The radiopharmaceutical composition of claim 20, wherein said tricarbonyl ^{99m}Tc coordination complex is [^{99m}Tc(CO)₃(MIBI)₃]⁺.

22. The radiopharmaceutical composition of claim 20 further comprising glucose and mannitol.
